(19) United States
(12) Patent Application Publication
(10) Pub. No.: US 2020/0268124 A1
(43) Pub. Date: Aug. 27, 2020

(54) DEVICE, SYSTEM, AND METHOD OF ERADICATING PARASITES

(71) Applicant: SPHINX SMARTHEAD TECHNOLOGIES LTD., Yoqneam (IL)

(72) Inventors: Doron Kenigsbuch, Shaarey Tikva (IL); Yechiel Kurtz, Haifa (IL); Gabi Horowitz, Kornit (IL)

(21) Appl. No.: 16/646,656

(22) PCT Filed: Sep. 14, 2017
(86) PCT No.: PCT/IL2017/051042

§ 371 Date: Mar. 12, 2020

(51) Int. Cl. A45D 24/30; A45D 24/32; A61L 2/00; A01M 3/00

(52) U.S. Cl. CPC A45D 24/30; A01M 3/007; A61L 2/0023; A45D 24/32

(57) ABSTRACT

A parasite eradicating device configured for eradicating parasites by producing a high-temperature airflow, directing it in a stream at an infestation area where parasites thrive, and suctioning the airflow away from the infestation area, such that the parasites are killed and their eggs rendered nonviable by the heat of the airflow.

### TECHNOLOGICAL FIELD

The present disclosure is concerned with the eradication of parasites, and more particularly with a device, system, and method for eradicating parasites.

### BACKGROUND ART

References considered to be relevant as background to the presently disclosed subject matter are listed below:
U.S. Pat. No. 5,261,427
U.S. Pat. No. 7,789,902 B2
U.S. Pat. No. 8,475,510 B2

Acknowledgement of the above reference herein is not to be inferred as meaning that these are in any way relevant to the patentability of the presently disclosed subject matter.

### BACKGROUND

In biology, a parasite is an organism that survives by living on another organism, the host. Ectoparasites, such as lice, fleas and some mites, are parasites which live on the outside of the host, for example, on the skin.

A louse is a wingless insect which lives on warm-blooded hosts, including humans. Head lice, body lice and pubic lice may suck blood from the host, or even chew the skin of the host.

Human head lice tend to live, feed, and lay their eggs (nits), within a distance of about 6 mm from the human scalp, where the conditions of temperature and humidity, as well as the proximity to their source of food is ideal for their survival and prosperity.

Female lice typically attach their eggs, the nits, to the base of the hair shaft within the 6 mm distance from the scalp. Nits hatch into nymphs and mature to become adult lice within a few weeks, at which point the female adult louse can lay eggs.

Head lice cannot hop or fly, but they can crawl very fast along a hair shaft. The greatest risk for exposure to infestation by head lice is head-to-head contact with a person who has a head lice infestation. Another risk, but a lesser one, is the shared use of personal items, such as bedding, towels, hats, scarves and hair brushes, as head lice can survive on a human host for approximately 30 days, but they generally cannot survive longer than 24 hours off the host.

Once a lice infestation begins, it can become full-blown very quickly, as the female louse can lay several hundred eggs during her 30-day life span. The lice and nits further prove to be very resilient and difficult to get rid of. Lice have six legs equipped with claws with which they grip a strand of hair tightly. The female louse lays each egg into a glue-like casing on the hair shaft which firmly cements each nit to the strand of hair. Neither the lice nor the nits are easily dislodged from the hair.

Over the years, no shortage of products, devices and methods have been devised to help humankind free itself of the scourge of the head louse, the most common of which include pediculicides (substances used to treat lice, e.g., shampoos containing an insecticide) and fine-toothed nit combs.

Studies have shown that over the years since the introduction of pediculicides to the lice-treatment market, lice have adapted and become resistant to some of the chemicals that once reliably killed them. Removal of lice and eggs from the head by fine-toothed comb is tedious, time-consuming, and often ultimately frustrating, as the efficacy of this treatment is highly dependent on the skill and meticulousness of the treatment provider.

Due to the difficulties of treatment, head lice infestations cause inconvenience to millions of people each year, particularly when they occur among kindergarten and school-age children among whom head lice infestations can spread quickly.

U.S. Pat. No. 5,261,427 discloses a lice comb device containing a blower heater, to heat and direct a stream of heated air toward a set of comb teeth attached to the device housing. US 2006/0130393 A1 discloses a method of eliminating an ectoparasite infestation that includes steps of defining a target area on an animal having an ectoparasite infestation, heating a volume of air, and applying the heated air to the target area with an airflow.

Another difficulty and source of frustration inherent in the use of conventional treatments for the eradication of parasites is the blindness with which the treatment provider finds himself or herself approaching the problem. The average person finds it exceedingly difficult if not completely impossible to see lice at the hair root level of an infestation area surface with the naked eye.

### GENERAL DESCRIPTION

According to a first aspect of the present disclosure there is provided a parasite eradicating device configured for eradicating parasites by producing a high-temperature airflow, directing it in a stream at an infestation area where parasites thrive, and suctioning the airflow away from the infestation area, such that the parasites are killed and their eggs rendered nonviable by the heat of the airflow.

Another aspect of the present disclosure is directed to a method for eradicating parasites by subjecting them to a high-temperature airflow stream.

In one embodiment, the parasite eradicating device can be a hand-held unit operable like a comb or brush, having at least two operational combing teeth extending from a housing, each operational combing tooth having a distal end configured to come into close proximity with a surface of the infestation area during a combing operation, and having at least one high-temperature airflow outlet nozzle out of which the high-temperature airflow can be propelled in a stream at the infestation area. The device is further configured to have at least one airflow intake aperture at least partially facing the at least one high-temperature airflow outlet nozzle, into which the airflow stream propelled from the at least one high-temperature airflow outlet nozzle can be suctioned away from the infestation area. The term close proximity suggests also contact with the treated surface.

An operational combing tooth is a tooth which contains at least one of a channel for conveying hot air under pressure to a high-temperature airflow outlet nozzle for emission, and a channel through which hot air is evacuated from an inlet aperture.

The arrangement is such that the high-temperature airflow, which exits the at least one high-temperature airflow outlet nozzle, is further organized into a concentrated stream of hot air by the suction force pulling it towards the facing airflow intake aperture, such that the concentrated stream of hot air slices through a separating space between the high-temperature airflow outlet nozzle and its facing airflow intake aperture.

Biologically, lice and nits cannot survive exposure to a blast of hot air for even a fraction of a second. When exposed to gradual heating, lice may have the opportunity to secrete hormones to increase their resistance to heat and their endurance in an overheated environment. In contrast, when lice and nits are exposed to a short pulse of extreme heat, their biological systems are overwhelmed by the mass of hot air, and they experience thermal shock. Applicants have observed, following experimentation with heat blast treatment on lice and nits, in post-mortem examination under a microscope, the effects of heat blast treatment; the shell of the louse shrinks and deforms, and the proteins in the body inside the shell undergo a process of coagulation, such as occurs to animal proteins cooked at high temperatures.

In an ordinary combing operation using the parasite eradicating device of the presently disclosed subject matter, lice and nits in an infestation area are exposed to a blast of hot air as the strands of hair on which they are located pass between the combing teeth of the device. The lice and nits are caught in the barrage of hot air propelled from each airflow outlet nozzle to its facing intake aperture in the spaces between the combing teeth. The lice are killed instantly or critically damaged by the blast of heat, and the nits are rendered non-viable, and will not hatch.

It will be appreciated that the term combing as used in this specification shall be understood to include any operation of combing through hair, including brushing, raking or plowing, in which a mass of strands of hair passing through the combing device, exits the combing device after being separated into small bundles of strands of hair, as a result of the combing teeth plowing through the mass of hair.

It will further be appreciated that the term combing tooth as used in this specification shall be understood to include any finger-like or prong-like protrusion from a hand-held device which can perform the operation of combing through hair described above.

In another embodiment of the presently disclosed subject matter, the parasite eradicating device can be a hand-held unit operable like a hair trimming machine.

A blast of hot air having a high temperature in a range between 80° C. and 120° C. can exterminate/damage a louse in a fraction of a second. The parasite eradicating device according to the present disclosure can be configured so that the amount of time that parasites and nits can be exposed to the high-temperature airflow during combing operation, is sufficient for extermination of the parasites and nits exposed to the stream of hot air.

To maximize efficacy, the barrage of hot air propelled from an airflow outlet nozzle to a neighboring intake aperture can occur in close proximity to the infestation area surface, such as a range of 0-6 mm from the infestation area surface, and practically, from the distal end of the respective combing tooth. In a parasite infestation, the greatest concentration of parasites and nits can be found in this range of distance from the infestation area surface, where the conditions of temperature and humidity, as well as the proximity to their source of food is ideal for their survival and prosperity.

The hand-held unit can therefore be configured so that the flow path of the stream of hot air crossing the separating space between each airflow outlet nozzle and its facing intake aperture is located within a distance of 0-6 mm from the infestation area surface, so as to encounter a maximum of parasite and nit targets.

The shapes and sizes of the of the airflow outlet nozzles can be varied in order to optimize the characteristics of the stream of hot air emitted from the nozzles. A number of nozzles of different shapes and sizes can also be grouped together, e.g., in line with one another, or in a different arrangement, in to optimize these characteristics.

The flow rate of the hot air stream emitted from the at least one airflow outlet nozzle can be 3-7 liters per minute.

In one example of the device, the separating space between the each airflow outlet nozzle and its facing airflow intake aperture can be in a range between 1-5 mm, with a range of 2-4 mm found suitable.

In another example of the disclosure, at least one high-temperature airflow outlet nozzle can be disposed on a side of a combing tooth, and its facing airflow intake aperture can be disposed on an adjacent combing tooth, at least partially facing the high-temperature airflow nozzle.

In yet another example, at least one high-temperature airflow outlet nozzle can be disposed on a side of a combing tooth, and its facing airflow intake aperture can be disposed at the shortest possible distance away on an adjacent combing tooth.

Each high temperature airflow outlet nozzle can be configured to direct the high-temperature airflow in a direction that is generally tangential to the surface of the infestation area, so that the parasites and eggs in close proximity to the surface of the infestation area are effectively targeted by the high-temperature airflow, while the surface of the infestation area, which can be sensitive to heat, e.g. a human scalp, is not heated beyond a threshold of discomfort with respect to heat as perceived by the host of the infestation, i.e. the treatment recipient, during a combing operation.

While the suction force applied through the intake apertures performs a principal function of organizing the hot air flow propelled from the airflow outlet nozzles into a concentrated stream of hot air, it can further perform a secondary function of evacuating excess heat away from the infestation area surface by evacuating air which may still be retaining some heat away from the infestation area surface. The suctioning of heat away from the infestation area surface in this manner can further ensure that while the high temperature airflow is adequately supplied at a sufficiently high temperature in sufficiently close proximity to the infestation area surface, a comfort level for the treatment recipient which is beneath the threshold of perceived discomfort can also be ensured.

The parasite eradicating device can be configured in additional ways to protect the treatment recipient from feeling any discomfort due to an excess of heat at the infestation area surface when undergoing heat blast treatment for eradicating parasites. In one embodiment, at least one spacer can be provided to maintain a distance between a distal end of the operational combing tooth and the infestation area surface.

A spacer can be a heat insulating element formed of a non-thermal conductive material, integral or integrated with an operational combing tooth and forming a separation between the operational combing tooth and the infestation area surface when the hand-held unit is brought as close as possible to the infestation area surface. The spacer can be integral with at least the distal end of the combing tooth, or integrated therewith.

In another embodiment, a spacer can be an auxiliary combing tooth, located in a position adjacent to an operational combing tooth, so that when the hand-held unit is brought as close as possible to the infestation area surface, the auxiliary combing tooth comes into contact with the infestation area surface, but the operational combing tooth does not come into contact with the infestation area surface.

A combing tooth as referred to hereinafter is directed to an operational tooth and an associated auxiliary tooth.

Methods of active cooling can also be employed in the parasite eradicating device of the presently disclosed subject matter to transfer heat away from the infestation area surface. Any known suitable method of active cooling can be employed, e.g., drawing heat away through contact with a highly thermally conductive material such as stainless steel, aluminum, nickel or titanium, optionally with the combination of circulation of a coolant fluid through tubing, TEC (thermoelectric cooling, also known as Peltier cooling) or heat pipe cooling.

For example, a spacer can be a cooling spacer, or it can be a thermally insulated spacer with at least a cooling portion cooled by an active cooling system, so that it feels cool to the touch, i.e., when contact is made between it and the infestation area surface. One example of such a spacer can be an element formed of a thermally conductive material, such as stainless steel, aluminum, nickel or titanium, and attached to an operational combing tooth, forming a separation between it and the infestation area surface when the hand-held unit is brought as close as possible to the infestation area surface. When the actively cooled spacer comes into contact with the infestation area surface, it can draw heat away from it, in addition to imparting a cool feeling to the treatment recipient.

In one example of such a spacer having active cooling, the spacer can be formed of tubing made of a thermally conductive material, surrounding the distal end of the operational combing tooth in close proximity thereto, and forming part of a closed circuit through which coolant fluid can flow and draw heat away from the infestation area surface. The cooling fluid can absorb heat as it flows in the tubing around the distal end of an operational combing tooth near the infestation area surface, and can dissipate heat over the distance it flows through the circuit after flowing away from the infestation area surface. Alternatively, heat can be actively removed at an area remote from the infestation area surface, and the cooling fluid can be re-cooled and recirculated by the cooling system.

In yet another embodiment, a spacer which is an auxiliary combing tooth can be cooled by an active cooling system.

Through experimentation, Applicants have realized that providing contact at the treatment surface area with actively cooled elements which are cool to the touch, can provide additional advantage beyond the objective advantage of a physical reduction in heat due to heat dissipation. A subjective advantage, of a psychological distortion of perception of heat, was discovered further to reports by treatment recipients of an enhanced feeling of relief from the heat of treatment when feeling, at the treatment area surface, cold spots alternating with hot spots.

Applicants have further realized that variation of the surface area of the cold spot can have a dramatic effect on the perception of heat relief perceived by the treatment recipient and that the actively cooled areas in the parasite eradicating device can be configured accordingly.

The hand-held unit of the parasite eradicating device provided in accordance with the presently disclosed subject matter can be light weight for ease of use. It can be self-contained, including all of the necessary elements for proper functioning of the device, or, in order to reduce its weight and increase its ease of use, some of the elements can be contained in a base unit, which can serve as a cradle for the device when it is not in use.

For example, the parasite eradicating device can comprise an air pump in order to provide positive air pressure for the flow of air propelled out of the high-temperature airflow outlet nozzles, as well as negative air pressure, i.e., suction, at the inlet apertures. The air pump can be located in the base unit, and both a positive pressure pipe as well as a negative pressure pipe can connect between the air pump in the base unit and a manifold in the hand-held unit. Positive pressure channels can channel the positive pressure air flow from the positive pressure chamber in the manifold to the high-temperature outlet airflow nozzles in the combing teeth. Negative pressure channels can channel the negative pressure flow from the inlet apertures to the negative pressure chamber in the manifold.

The suction flow rate generated by the air pump can be configured to substantially match the positive flow rate of the hot air stream emitted from the at least one airflow outlet nozzle so that the flow of the hot air stream is orderly and does not become diffused in the ambient space near the separating space between the at least one high-temperature airflow outlet nozzle and the at least one airflow intake aperture.

In another example of the device, the flow rate and the temperature of the hot air stream can be adjustable by a user within a range allowed by the device. Likewise the intake flow rate can be adjustable so as to control the evacuation of heat from the infestation area surface.

The parasite eradicating device can comprise additional elements necessary for its functioning, and these elements can be located in the hand-held unit or in the separate base unit.

A power supply unit in the device can be connected by a power cable to an ordinary power supply outlet, such as a power supply outlet in a residence. The power supply unit can convert the power from the provided household voltage to the low voltage required to operate the device. The power supply unit can then supply power to the units in the device which require power, e.g., the air pump, air heating system, and active cooling system.

In order for the parasite eradicating device to begin operation, power is supplied to the air pump to begin the flow of air, and to the heating system to begin heating the air. One method for heating the air can be the use of a heating coil located in the air supply pipe connecting the air pump in the base unit and the manifold in the hand-held unit. In another embodiment, individual heating coils can be located in or adjacent each positive pressure channel of each combing tooth having a high-temperature airflow outlet nozzle, or at a cluster of operational combing teeth. In yet another embodiment, recycled heat from other systems in the device, e.g. the active cooling system, can be used to heat the air.

The parasite eradicating device in accordance with the presently disclosed subject matter can further comprise one or more sensors, a processor and a controller to ensure that the parasite eradicating treatment provided by the parasite eradicating device is as effective and as safe as possible. That is, using sensors, and in response to the data provided by the sensors, using algorithms, the controller can adjust the power supplied to the air pump and to the heating system to ensure that the hot air flow emitted from the high-temperature airflow outlet nozzles is hot enough and robust enough to exterminate the parasites and their eggs, but not too hot to cause discomfort to the host of the infestation, i.e., the treatment recipient. For example, the power supplied to the air pump can be adjusted so as to adjust the supplied air flow rate.

Temperature sensors, which can be in the form of thermocouples, can be provided at the distal ends of the operational combing teeth, spacers or auxiliary combing teeth, on a surface which comes into contact with the infestation area surface.

Additional temperature sensors, which can also be in the form of thermocouples, can be provided in the negative pressure channels opposite the inlet apertures to provide another indication of the temperature in the treatment area, i.e., at the infestation area surface in the vicinity of the operational combing teeth.

Alternatively, temperature sensors can be provided to measure the temperature of the airflow exiting the high-temperature airflow outlet nozzle.

Yet another temperature reading, of the scalp temperature, can be provided using remote (contactless) IR technology.

Applicants have realized that it is important to collect data from multiple sources in order to assess the temperature in the treatment area since it can be influenced by factors other than the power input to the air pump and the air heater which can be controlled to adjust (increase or decrease) air flow rate or heat. For example, density of hair, i.e., how thick the hair is in the treatment area, can affect how much heat is trapped in the treatment area, and for thicker hair, it could be necessary to reduce the hot air flow in order for the temperature at the treatment area to not rise to uncomfortable levels.

When a detected temperature is determined to be higher than a pre-defined threshold value, the device can be configured to respond in such a way as to reduce the temperature at least where the temperature was detected to be above an allowable threshold value.

The temperature can be reduced by reducing the heat being delivered to the device in the vicinity of the temperature sensor. This can be accomplished, for example, by causing an automatic shut-off of the device, or by causing the heating element to turn off. In another example, the temperature sensed by the temperature sensor can be reduced by increasing the heat removal rate in the vicinity of the temperature sensor. This can be accomplished, for example, by causing an increase in the rate of suction flow suctioning hot air through at least the airflow intake aperture disposed at the location of the temperature sensor where the temperature was detected to rise above the allowable temperature threshold.

In another embodiment, the high-temperature airflow outlet nozzles can be configured so that the hot air flow can be diverted in a direction opposite the treatment surface area as a safety measure.

Likewise, when a detected temperature is determined to be lower than a pre-defined threshold value, the device can be configured to respond in such a way as to increase the flow of hot air at least where the temperature was detected to be below a minimum threshold value necessary for treatment. In another exemplary scenario, if the temperature remains lower than a pre-defined threshold value for a period of time exceeding a pre-defined duration, the device can be configured to shut down and to alert the user of an apparent malfunction preventing the temperature of the airflow from rising high enough so that effective treatment can be provided. Device can be preset to 3-6 treatment levels as option for the user to pre-define the treatment level of delivered energy (temperature and flow rate) to kill lice (aggressive, effective or gentle etc.)

The hand-held unit may further have motion sensing capabilities to detect motion of the hand-held unit with respect to a treatment area surface. Motion sensing capabilities can be provided in the hand-held unit by inclusion of, for example, motion sensors, optics and PCBs.

Motion sensing capabilities can provide advantages with respect to efficacy by keeping track of motion related data such as the speed of combing, and how much distance is covered over the infestation area surface during treatment. This information can then be provided to the user as treatment feedback, enhancing the user experience and contributing to increased efficacy of treatment.

For example, the user can be notified in real time whether the speed of combing during a treatment is too fast or too slow. At the end of treatment, the user can be advised if the infestation surface was thoroughly combed, if the treatment should be resumed, or repeated after a certain number of days based on a recommended treatment protocol that can be included in an algorithm.

Such treatment feedback can be provided to the user, e.g. via a GUI, on the hand-held unit or base unit of the parasite eradicating device. With the addition of a communications component in the base unit, for example, treatment feedback can be sent to a user's smartphone. The user experience could then further be enhanced by connecting the user to a community of parasite eradicating device users, via an application for smartphones.

Motion sensing capabilities can also provide advantages with respect to safety, for example, by ensuring that full hot air jet capacity is enabled only when movement of the hand-held device is detected with respect to the infestation area surface. The determination of the distance between the combing teeth and the infestation area surface can be accomplished, for example, by using optics to analyze how projected light is scattered back from the surface to the sensor.

In another embodiment of the device, a mechanical arrangement can be employed to ensure that full hot air jet capacity is enabled only when movement of the hand-held device is detected with respect to the infestation area surface. In this embodiment, each one of the combing teeth can be configured with a retraction mechanism, disposed between the combing tooth and the housing of the hand-held device. The retraction element can be configured to be normally biased such that its combing tooth is in a normally extended position and such that the combing tooth retracts under pressure exerted upon it by the infestation area surface, when a combing operation begins.

A first advantage provided by the configuration of each of the combing teeth with a retraction element can be the enablement of the device to be more effectively used along a curved or non-linear or non-planar region, e.g., the human scalp, by allowing each combing tooth to independently follow the curve of the treatment area surface beneath it.

A second advantage provided by the configuration of each of the combing teeth with a retraction element can be the mechanical means mentioned above to ensure that hot air jet capacity is enabled only when movement of the hand-held device is detected with respect to the infestation area surface. That is, the hot air jet capacity can be enabled only when some or all of the retraction elements are compressed beyond a pre-defined threshold value. Alternatively, or in combination, one or more pressure sensors can be provided for sensing pressure over the infestation area surface, such that the hot air jet capacity can be enabled only when some or all of the pressure sensors are activated beyond a pre-defined pressure threshold value.

The parasite eradicating device can have a stand-by mode when the device has been powered on, but has not yet been activated. The device may be kept in stand-by mode when certain safety prerequisite conditions have not been met, for example, when movement of the device with respect to the treatment area surface has not been detected, as described above.

In stand-by mode, the device may be in a status in which it is ready to work. In stand-by mode, air can be heated at a low flow rate.

The hand-held unit may further have air flow sensing capabilities to detect whether there has been an interruption to the air flow through the device, for example, due to a blockage in an air flow channel. The hand-held unit can be configured to have filters to prevent debris, e.g., dandruff flakes, from blocking air flow channels in the device. Filters can be provided at the airflow intake apertures and can be removable, maintainable and/or replaceable. Alternatively, a filter or filters, which can be similarly removable, maintainable and/or replaceable can be provided at a more central location or locations of the air flow channel and pipe network in the device.

The processor and controller of the device, responding to data, such as temperature and motion, provided by the sensors of the device, and processed according to algorithms, can automatically adjust the air heating rate, positive pressure flow rate, suction flow rate and coolant fluid flow rate, in order to provide parasite eradicating treatment in the most effective and safe manner possible.

The combing teeth of the device can be widely spaced in order to facilitate combing, encourage ease of use, and repeated use. Auxiliary combing teeth, which encounter the hair first in a combing operation, can have an optimized functional design such as a fin-shaped design with tapered edges, to lift and separate the hair as it is guided into the hot air stream in the treatment zone between the operational combing teeth. This can enable optimized penetration of the distal ends of the combing teeth into the 0-6 mm zone above the treated surface, thus maximizing the number of lice and nit targets intercepted and eradicated by the hot air stream emitted at the distal ends of the teeth, and thus maximizing the efficacy of the device.

On account of the wide spacing of the combing teeth, and their design optimized for lifting and separating hair, the device can be used on any hair without any significant pre-treatment, e.g., tangled hair.

In another embodiment, the combing teeth can be designed to move, such as in a vibrating motion, or in a rotating motion, in order to detangle tangles in the hair and to expose a greater amount of hair to heat at a single combing stroke.

In another embodiment, the combing teeth can be covered by disposable sleeves made of thin, pliable material such as silicone, to allow sterile use for multiple treatment recipients, and to filter the suctioned air. The sleeve can be impermeable and configured with apertures at respective outlet and inlet locations of the combing teeth, or it can be made of fine screen material facilitating airflow therethrough.

In another embodiment, the hot air flow can be emitted in multiple directions from a combing tooth. In one example, hot air flow can also be emitted in a direction of the combing from a nozzle on a forward and or backward facing facet of a combing tooth.

In one example of the parasite eradicating device, the hot air can be saturated with liquid vapor, in a range of 50% to 100% saturation, thereby maximizing the effect of the thermal shock on the target lice and nits. The liquid can be water, or other anti-lice treatment substances such as rosemary oil, tea tree oil, or hair/skin treating agents, etc.

In additional embodiments, the device can be used as a hair dryer, a hair ironing device for straightening hair, hair styling, and a scalp massager.

In yet another embodiment of the presently disclosed parasite eradicating device, the device can be equipped with an image recognition system comprising imaging capabilities, data collection capabilities and processing capabilities in order to implement computerized vision and/or digital imaging technologies, including digital image analysis, to determine whether an area that can potentially be infested with parasites is in fact infested with parasites, by detecting the presence of parasites or nits in the area.

In yet another embodiment of the presently disclosed parasite eradicating device, the image recognition system can be further employed to facilitate the eradication of parasites and nits present in a parasite infestation.

Computerized vision and digital imaging technologies, which address the problem of blindness by providing information and guidance to a treatment provider regarding what cannot be seen with the naked eye, can be very advantageous when applied to the problem of parasite eradication.

For example, the device can provide indicia to a user, the treatment provider, whether a combed area is ‘infested’, when the presence of parasites or their eggs are detected during a combing operation, or ‘clear’, when the presence of parasites or their eggs are not detected during a combing operation.

In another example, the device can guide a user, to sub-areas of the infestation area where there are remaining lice and nit targets, rather than sub-areas of the infestation area which have already been cleared of lice and nit targets. Thus the image recognition system provides the user with feedback verifying whether or not the treatment operation is indeed successful, or to what degree it is successful. The image recognition system could be also serve for diagnostic purposes (i.e. detecting the presence of parasites), also at the event that the treating mechanism is inactive.

### BRIEF DESCRIPTION OF THE DRAWINGS

In order to better understand the subject matter that is disclosed herein and to exemplify how it may be carried out in practice, embodiments will now be described, by way of non-limiting examples only, with reference to the accompanying drawings, in which:

FIG. 1 is a top perspective view of a hand-held unit of a parasite eradicating device according to one example of the presently disclosed subject matter;

FIG. 2 is an enlarged bottom view of the device head of the hand-held unit shown in FIG. 1;

FIG. 3A is a perspective side view of the device, oriented so that the distal faces of the combing teeth can be seen;

FIG. 3B is a perspective side view of the device head shown in FIG. 2 and FIG. 3A, oriented so that the proximal faces of the combing teeth can be seen;

FIG. 4 is an end view of the proximal end of the hand-held unit shown in FIG. 1;

FIG. 5 is a perspective view of the hand-held unit shown in FIG. 1, shown with its base unit;

FIG. 6 is a perspective top view of the device head;

FIG. 7 is a perspective view of a manifold and set of combing teeth, for use in a hand-held unit of a parasite eradicating device according to a second example of the presently disclosed subject matter;

FIG. 8 is a side view of the manifold and set of combing teeth shown in FIG. 7;

FIG. 9A is a perspective bottom view of the manifold and set of combing teeth shown in FIG. 7, oriented so that the left-side faces of the combing teeth can be seen; and

FIG. 9B is a perspective bottom view of the manifold and set of combing teeth shown in FIG. 7, oriented so that the right-side faces of the combing teeth can be seen; and

FIG. 10 is a perspective view of the hand-held unit and its base unit according to another example of the presently disclosed subject matter.

### DETAILED DESCRIPTION OF EMBODIMENTS

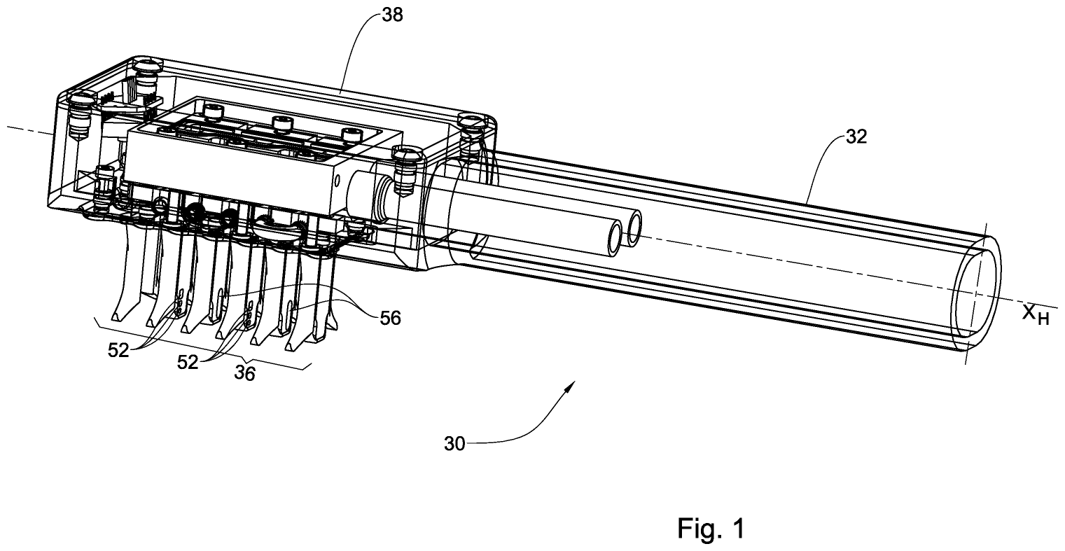

Reference is now made to FIGS. 1-6, which illustrate a hand-held unit 30 of a parasite eradicating device 10 according to one embodiment of the presently disclosed subject matter.

The hand-held unit 30 is operable like a hair brush or comb, having a handle 32 configured for holding manually, and a device head 38 at the distal end of handle 32. As shown in FIG. 1, the handle 32 is oriented along a longitudinal axis X_{H} of the device 10.

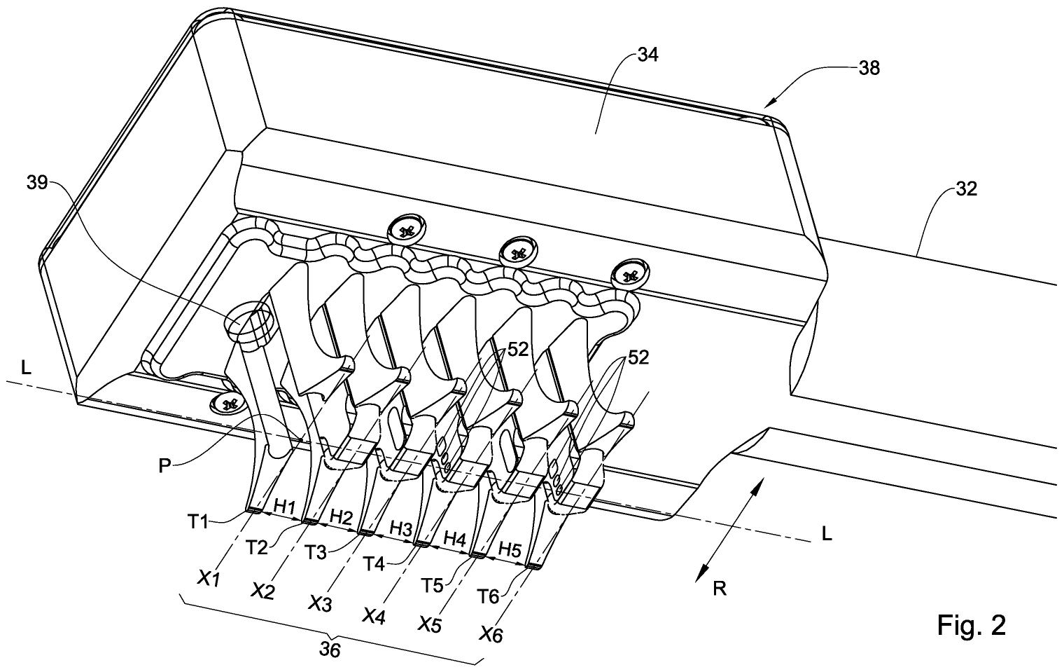

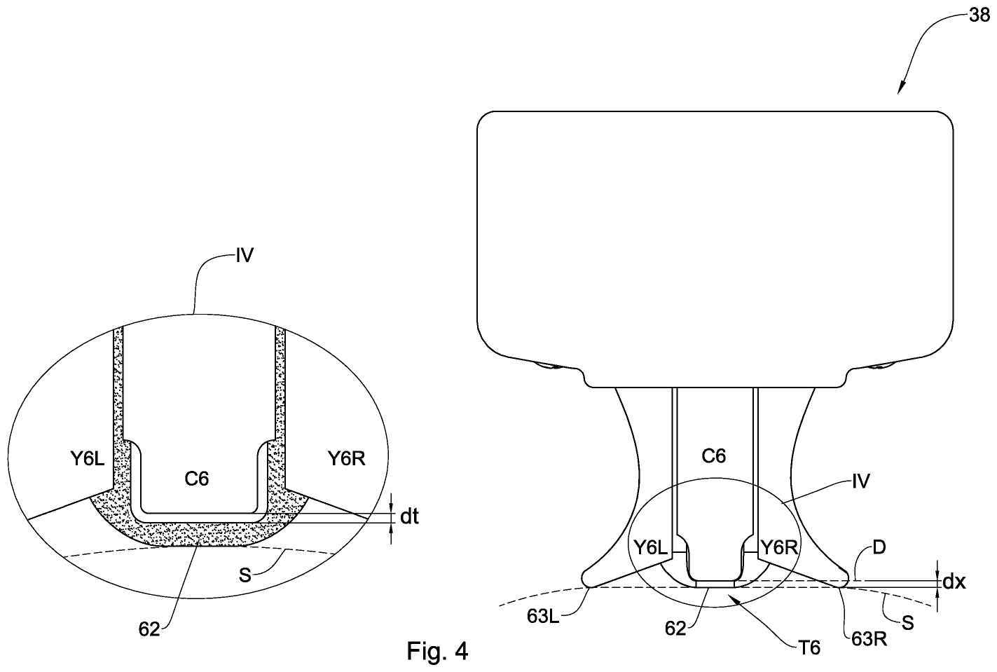

The device head 38, an enlarged view of which is shown in FIGS. 2, 3A, 3B and 6, comprises a housing 34 and a set of combing teeth 36. The set of combing teeth 36 is configured to be passed through a section of hair, in an ordinary combing motion, along a surface from which the hair grows where a parasite infestation has been detected, such as along a scalp surface of a human head of hair which is infested with lice. This surface, labeled S, is shown in FIG. 4.

In accordance with the presently disclosed subject matter, lice and their nits are eradicated during a treatment performed using device 10, in which the lice and nits are subjected to high-temperature air flow, emitted out of an at least one high-temperature airflow outlet nozzle 52 located on a side of at least one combing tooth T.

On a side of a neighboring combing tooth T, at least partially facing the high-temperature air flow emitted out of the high-temperature airflow outlet nozzle 52, a suction force is exerted into an inlet aperture 56, which suctions the high-temperature air flow into it. Suctioning of the high-temperature air flow emitted out of the high-temperature airflow outlet nozzle 52 into the inlet aperture 56 accomplishes two objectives.

Firstly, the suction force serves to organize the high-temperature air flow into a concentrated stream, which then flows forcefully across the distance which separates the inlet aperture 56 from the high-temperature airflow outlet nozzle 52, in the space H between the combing teeth T.

Secondly, the suction force serves to remove heat form the infestation surface area, where a buildup of excessive heat could cause discomfort to the host of the parasite infestation, i.e., the recipient of the parasite eradicating treatment performed by device 10.

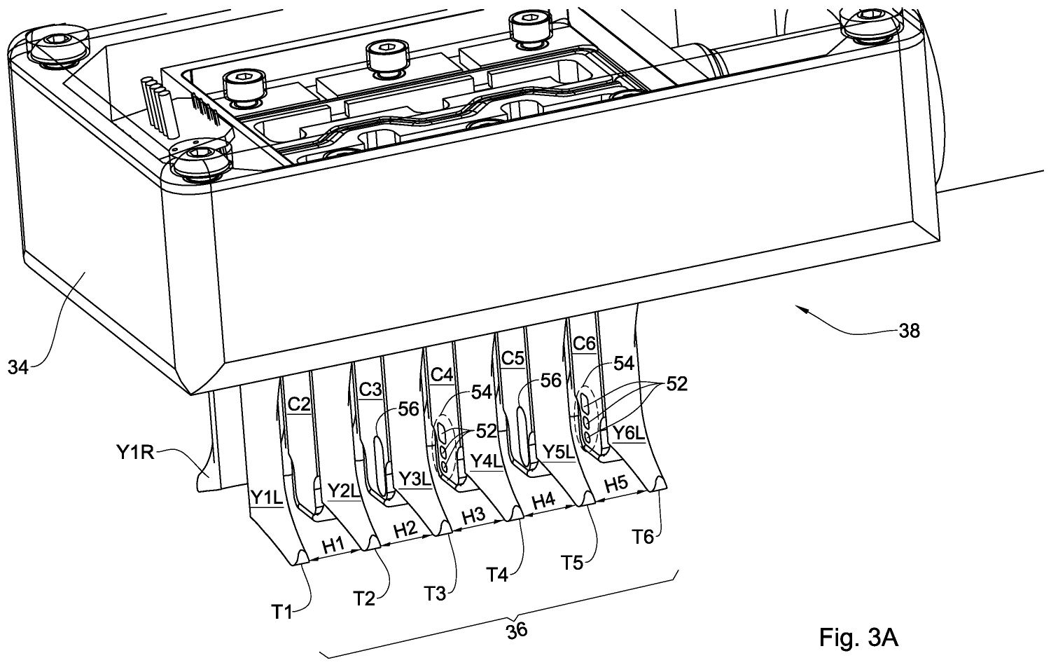

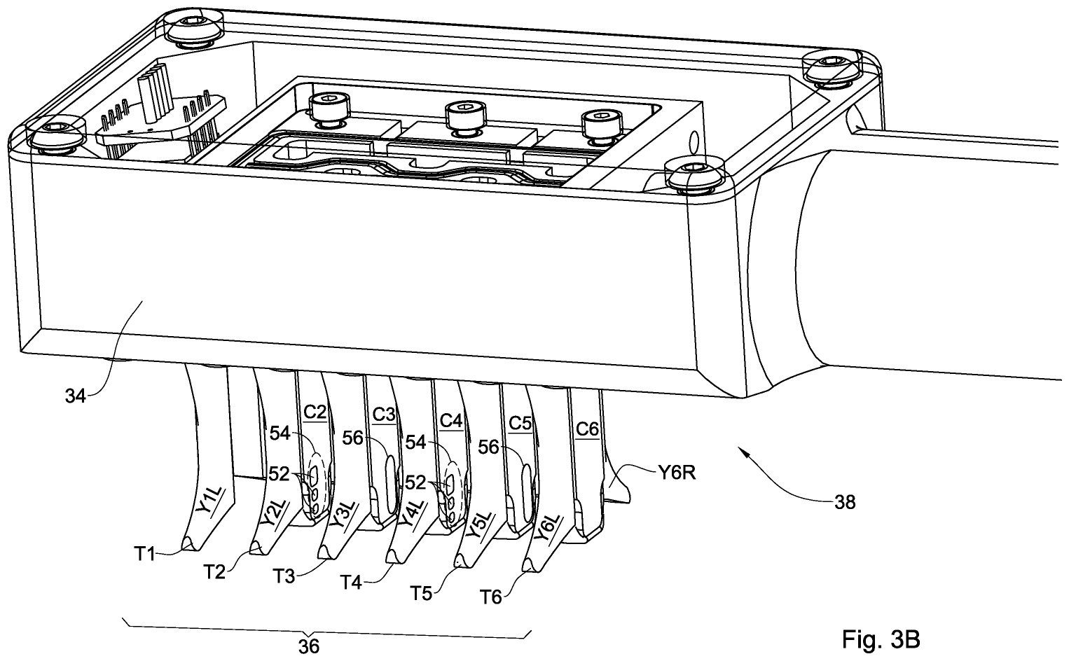

As illustrated in FIGS. 2, 3A and 3B, set of combing teeth 36 has six combing teeth T1 through T6, where combing tooth T1 is located at the distal extremity of the set of combing teeth 36, and combing tooth T6 is located at the proximal extremity of the set of combing teeth 36.

It will be appreciated that a combing tooth T can be shaped so as to be functional in terms of performing a combing operation, while not necessarily having the functionality of emitting or suctioning hot air. For example, combing tooth T1, illustrated in FIGS. 2, 3A and 3B, is a motion sensing combing tooth configured for sensing movement of the device 10, while not being configured for emitting or suctioning hot air.

It will be further appreciated that sensors of various types, such as temperature sensors, can be provided on a combing tooth T which does have the functionality of emitting or suctioning hot air.

Each combing tooth T1-T6 has a respective longitudinal axis X1-X6 as shown in FIG. 2. It will be appreciated that the longitudinal axes X1-X6 are oriented in a transverse direction with respect to the longitudinal axis X_{H} of the device 10, shown in FIG. 1.

As indicated by arrow R shown in FIG. 2, hand-held unit 30 is configured for combing in either direction along a line parallel to the longitudinal axes X1-X6 of combing teeth T1-T6. Thus, during combing, strands of hair pass in the combing paths H1-H5 formed by the spaces between the combing teeth T1-T6.

Human head lice tend to live, feed, and lay their eggs (nits), within a distance of about 6 mm from the human scalp, where the conditions of temperature and humidity, as well as the proximity to their source of food is ideal for their survival and prosperity. Therefore, hand-held unit 30 and combing teeth T are configured so that when device head 38 is brought as close as possible to the infestation area surface, such that contact is made between the most distal contact points of combing teeth T and the infestation area surface, the stream of hot air flowing between each high-temperature airflow outlet nozzle 52 and its facing inlet aperture 56 is propelled along a flow path located between 0-6 mm from the infestation area.

As shown in FIGS. 1, 2, 3A and 3B, each of combing teeth T2-T6 is comprised of three components. Combing tooth T1 is a sensing combing tooth, the function of which will be discussed below in further detail. In the end view of hand-held unit 30 shown in FIG. 4, a side view of the three components comprising combing tooth T6 can be seen.

The central part of each combing tooth T2-T6, is a central operational combing tooth C2-C6, respectively, as shown in FIGS. 3A and 3B. Central operational combing tooth C6 of combing tooth T6 can also be seen in FIG. 4. Central operational combing teeth C2-C6 can be seen to be lined up in a row along central axis L shown in FIG. 2, which is substantially parallel to the longitudinal axis X_{H} of the device 10, shown in FIG. 1.

Each central operational combing tooth C is flanked on both of its sides, along longitudinal axis X of the combing tooth T, by an auxiliary combing tooth Y. In FIG. 4, auxiliary combing teeth Y6R and Y6L can be seen to be flanking operational combing tooth C6 on its right and left sides respectively. In FIGS. 3A and 3B, the auxiliary combing tooth Y1L of sensing combing tooth T1, and the auxiliary combing teeth Y2L-Y6L, which flank operational combing teeth C2-C6, respectively, on their left side, can be seen. Furthermore, in FIG. 3A, the auxiliary combing tooth Y1R of sensing combing tooth T1 can also be seen. In FIG. 3B, the auxiliary combing tooth Y6R of sensing combing tooth T6 can also be seen.

Auxiliary combing teeth Y can be seen to be fin-shaped with tapered edges and rounded end points. The purpose of this shape is to separate and lift the hairs in the infestation area as they are combed with the set of combing teeth 36, thereby guiding the hairs into the combing paths H and facilitating effective treatment of the 0-6 mm zone near the root of each hair.

As shown in FIGS. 3A and 3B, high-temperature airflow outlet nozzles 52 and inlet apertures 56 are disposed at the distal ends of operational combing teeth C where, when device head 38 is brought as close as possible to the infestation area surface and the most distal extremity points of exemplary combing teeth T come into contact with the infestation area surface, high-temperature airflow outlet nozzles 52 and inlet apertures 56 will be located in the 0-6 mm zone from the infestation area surface.

Operational combing teeth C can be considered to be hot in temperature, as heated air flows through them and therefore, in order to protect the treatment recipient from discomfort due to heat, the operational combing teeth C are prevented from coming into contact with the infestation area surface.

This can be done in a variety of ways, including the attachment of spacers to the distal ends of the operational combing teeth, for example, or by provision of at least one auxiliary combing tooth, located in a position adjacent to an operational combing tooth, and configured to come into contact with the infestation area surface when the hand-held unit is brought as close as possible to the infestation area surface, such that the operational combing tooth does not come into contact with the infestation area surface.

As shown in FIG. 4, when device head 38 is brought as close as possible to the infestation area surface S, contact is made with infestation area surface S by distal extremity points 63R and 63L of auxiliary combing teeth YR and YL respectively, while the distal end of operational combing tooth C6 remains at the level indicated by the letter D, at a distance dx from infestation area surface S.

In the example shown in FIG. 4, the distal end of operational combing tooth C is further prevented from coming into contact with infestation surface S by spacer 62. In the embodiment of parasite eradicating device 10 illustrated in FIG. 4, spacer 62 is a flattened section of tubing, constructed of highly thermally conductive material for drawing heat from infestation surface S when in contact with it. The tubing can be seen in FIG. 4 to bend around the edges of operational combing tooth C6 and continue upwards, forming part of a complete circuit of tubing through which coolant fluid flows. The tubing envelops each one of operational combing teeth C2-C6, thus providing active cooling to the entire set of combing teeth 36 of the device 10.

It will be appreciated that spacer 62 is an actively cooled spacer and is cool to the touch, while operational combing tooth C is hot, so it is preferable that device 10 is configured such that they do not come into contact, in order that each can retain its own thermal properties to the greatest extent by being isolated from one another. However, it is also necessary that the distal end of operational combing tooth C come into very close proximity with infestation area surface S. Therefore, as best illustrated in FIG. 4 and in particular in the zoomed out portion designated IV in that figure, spacer 62 is configured to be very thin, preferably less than 1 mm in thickness, and being in close proximity thereto, spaced dt from the distal end of the combing tooth. The tubing of which spacer 62 is formed is flattened to an extreme extent, while a thin space remains within through which the coolant fluid can flow.

In the embodiment of parasite eradicating device 10 illustrated in FIGS. 1-6, seen most clearly in FIGS. 3A and 3B, high temperature airflow outlet nozzles 52 are grouped in sets 54 of three nozzles 52 each, and two such sets 54 can be seen in FIG. 3A to be disposed on the distally facing sides of operational combing teeth C4 and C6. In FIG. 3B, another two such sets 54 of three nozzles 52 each can be seen on the proximally facing sides of operational combing teeth C2 and C4. It can consequently be understood that operational combing tooth C4 has high-temperature airflow outlet nozzles 52 on both of its faces which are parallel to the longitudinal axis X4 of combing tooth T4.

Two inlet apertures 56 can be seen in FIG. 3A to be disposed on the distally facing sides of operational combing teeth C3 and C5. In FIG. 3B, another two inlet apertures 56 can be seen on the proximally facing sides of operational combing teeth C3 and C5. It can consequently be understood that both operational combing teeth C3 and C5 have inlet apertures 56 on both of their faces which are parallel to the longitudinal axes X3 and X5 of combing teeth T3 and T5 respectively.

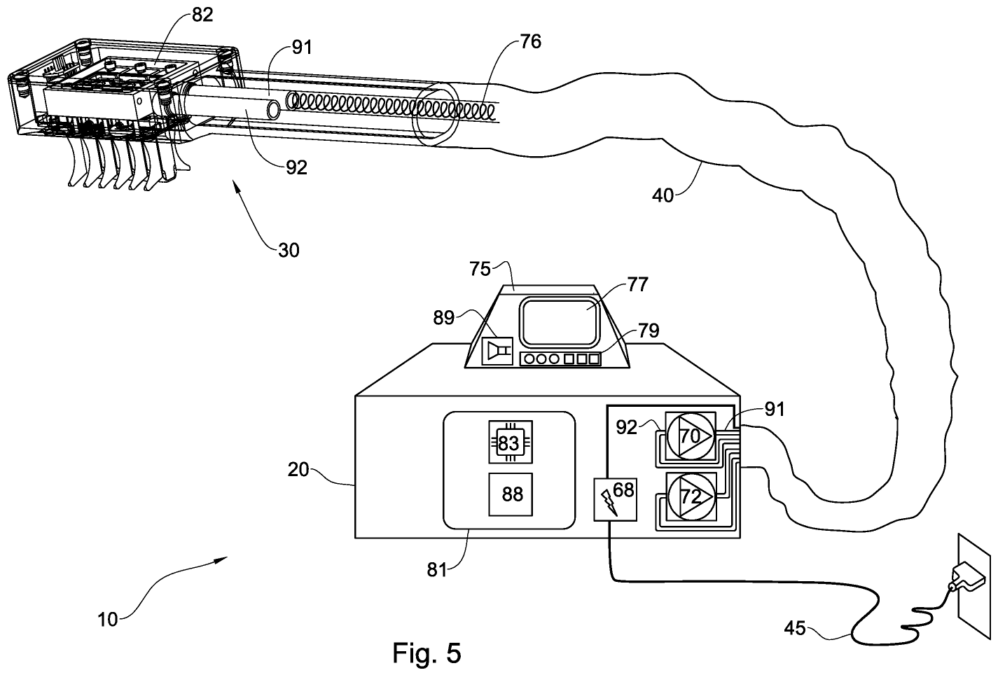

In FIG. 5, hand-held unit 30 of parasite eradicating device 10 is shown to be connected by flexible tubing 40 to a base unit 20. In the embodiment of the device 10 shown in FIGS. 1-6, the device 10 comprises a hand-held unit 30, shown in FIGS. 1-6, as well as a base unit 20, shown in FIG. 5. Some of the elements of the device 10 which can operate remotely from the hand-held unit 30, via connections through the flexible tubing 40, can be located in the base unit 20, thus keeping the weight of the hand-held unit 30 to a minimum and thereby increasing its ease of use. The base unit 20 can further serve as a cradle for the hand-held unit 30 when it is not in use. Tubing 40 can be detachable at either of its ends.

As shown in FIG. 5, the base unit 20 comprises a power supply unit 68, an air pump 70 and a coolant fluid pump 72. The base unit 20 further comprises a control panel 75, which can comprise a screen 77 and control buttons 79. In another embodiment, control panel can have fewer or no control buttons 79, and screen 77 could be a touch-screen which could be used to control the device 10.

The power supply unit 68 is shown to be connected by a power cord 45 to an ordinary power supply outlet, such as is located in a residence or commercial establishment. The power supply unit 68 converts the power from the provided household voltage to the low voltage required to operate the device 10. The power supply unit 68 supplies power to the units in the device 10 which require power, e.g., the air pump 70, the air heating system, and the active cooling system.

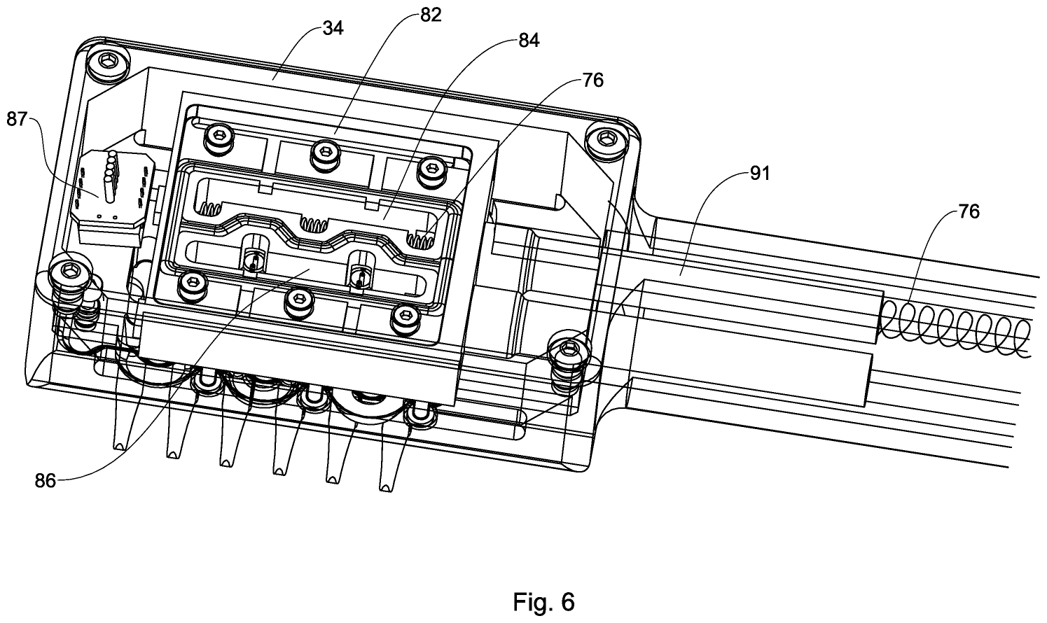

In order for device 10 to begin operation, power is supplied to the air pump 70 to begin the flow of air, and to the heating system to begin heating the air. One method for heating the air can be the use of a heating coil 76 located in the air supply pipe 91 connecting the air pump 70 in the base unit 20 and the manifold 82 (shown in FIG. 6) in the hand-held unit 30.

In another embodiment, individual heating coils can be located in each positive pressure channel of each combing tooth having a high-temperature airflow outlet nozzle. In yet another embodiment, recycled heat from other systems in the device, e.g. the active cooling system, can be used to heat the air.

The air pump 70 provides both positive air pressure for the flow of air propelled out of the high-temperature airflow outlet nozzles 52, as well as negative air pressure, i.e., suction, at the inlet apertures 56. As shown in FIGS. 5 and 6, the air pump 70 is connected to a positive pressure manifold chamber 84 in the manifold 82 via an air supply pipe 91. The air pump 70 is also connected to a negative pressure manifold chamber 86 in manifold 82 via a negative pressure (suction) pipe 92.

The positive pressure hot air flow is channeled from the positive pressure manifold chamber 84 to positive pressure hot air channels in the operational combing teeth C having high-temperature airflow outlet nozzles 52. Negative pressure hot air flow (suction) is channeled from the intake apertures 56 through negative pressure (suction) hot air channels in the operational combing teeth C (which have intake apertures 56), to negative pressure manifold chamber 86, and then through negative pressure pipe 92 back to air pump 70.

Base unit 20 in FIG. 5 is further shown to comprise a coolant fluid pump 72 for pumping the coolant fluid for the active cooling system of the device 10, of which the spacer 62, shown in FIG. 4 forms a part. As further shown in FIG. 5, the coolant fluid pump 72 has a coolant fluid outflow pipe 93, out of which relatively cool coolant flows towards hand-held unit 30, and a coolant fluid intake pipe 94, into which relatively heated coolant flows back into the coolant fluid pump 72 after being circulated around the operational combing teeth C2-C6 in set of combing teeth 36.

The flexible conduit 40 is configured to be of sufficient diameter to allow the passage through it of the wires and tubes required for connections between elements in the hand-held unit 30 and the base unit 20, e.g., electrical and communications wiring for power and signal transmission, the air supply pipe 91, the suction pipe 92, the coolant fluid outflow pipe 93, and the coolant fluid intake pipe 94.

As mentioned above, combing tooth T1, illustrated in FIGS. 2, 3A and 3B, is a motion sensing combing tooth configured for sensing movement of the device 10 with respect to a treatment area surface. Motion sensing can be provided in device 10 by any suitable method known in the art. In the example of device 10 shown in FIGS. 1-6, motion sensing is enabled by an optics assembly 39, which, as illustrated in FIG. 2, is located at an opening in housing 34 which is in line with an imaginary point P where central axis L intersects longitudinal axis X1 of combing tooth T1. Information from the optics assembly is then transferred to a PCB 87 (which is shown in FIG. 6) for generating a signal responsive to motion.

Base unit 20 in FIG. 5 is further shown to comprise a computing unit 81, which comprises a processor 83 and a controller 88. The processor 83 can process the signals received from the sensors in device 10, and the controller 88 can adjust the operating parameters of the device 10 accordingly. For example, the controller 88 can adjust the power supplied to the air pump and to the heating system to automatically shut off the flow of hot air in response to a signal received from the processor 83 indicating that an automatic shut-off of the device is required to ensure the comfort or safety of the treatment recipient.

Base unit 20 in FIG. 5 is further shown to comprise a speaker 89. The controller 88 of device 10 can convey treatment or operational information, guidance or warnings to a user in an audible manner (e.g., “beep” sounds, or pre-recorded speech message) via audio speaker 89. Alternatively, messages can be conveyed visually, (e.g., displayed on display unit 77, or via one or more LEDs or other visual indicator).

Processor 83 may track, log, or otherwise supervise the treatment along a time-line, such as, within single usage session and/or across multiple usage sessions (e.g., across multiple hours or days or weeks) on the basis of data provided by the sensors of device 10.

Controller 88 may ensure safety of the user that operates the device 10 and/or safety of the user that is being treated by the device 10; for example, by utilizing one or more temperature sensors; detection or sensing of device movement relative to the scalp or the head or the hair; ensuring that hot air is blown away from the scalp and/or in a direction that is tangent to the scalp or parallel to the scalp (and not towards the scalp, directly or in a slanted manner); a mechanism or activator/deactivator controller, to enable full hot-air jet capacity through nozzles or air-outlets, only when all (or most) of the teeth are activated by physical pressing on the scalp (e.g., by using springs or membranes or flexible intermediate elements); mechanism to enable a “stand-by” mode when the device is powered, but not yet activated, in order to keep the device ready to operate with hot-air output at full air jet capacity, and such that during the stand-by mode the air is optionally heated with low capacity (e.g., lower temperature, and/or lower air speed).

In some embodiments, the user safety controller 88 and/or other related sensors or controllers (e.g., movement motion or movement sensor), are used for safety measures or safety purposes. For example, the jet of hot air will ceased or stopped or paused, if the device is not moving relative to scalp/hair, or is not moving at all, or is not moving for a pre-defined time period (e.g., for at least 1 second, or for at least 2 seconds, or for at least K seconds wherein K is a positive number). In some embodiments, a temperature sensor may be used for safety measures or safety purposes; for example, the jet of hot air is ceased or stopped or paused if the contact tip(s) temperature is too high, or is greater than a pre-defined threshold value; thereby detecting a possible failure in the cooling system and/or the heat evacuation system of the device. It is noted that portions herein, that relate to a spring or flexible mechanism being Shrunk or Compressed, may further comprise a state of such spring or mechanism being “loaded” or “armed” or “triggered” or “pressed”.

Optionally, controller 88 may take into account data that is sensed or acquired or measured by one or more sensors which may be included in device 10 or which may be operatively associated with device 10; for example, temperature sensor, humidity sensor, accelerometer(s), gyroscope(s), orientation sensors, positioning sensors, compass, illumination level sensor, acoustic sensors, or the like.

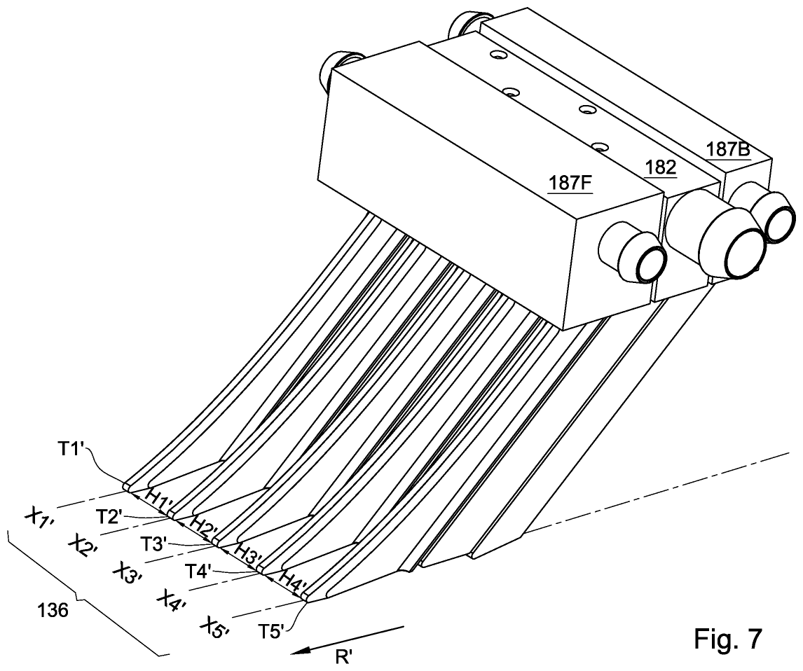

An alternative embodiment of the assembly comprising the combing teeth set 36 and the manifold 82 is shown FIGS. 7, 8, 9A and 9B. Corresponding elements of the alternative embodiment have been numbered similarly to the elements with which they correspond in the first embodiment. The reference numerals of the alternative embodiment have been increased by 100 with respect to the reference numerals of the first embodiment, or appended with a prime (′) symbol.

As shown in FIG. 7, set of combing teeth 136 of the alternative embodiment has five combing teeth T1′ through T5′.

Each combing tooth T1′-T5′ has a respective longitudinal axis X1′-X5′ as shown in FIG. 7. As indicated by arrow R′ shown in FIG. 7, the set of combing teeth 136 is configured for combing in one direction only along a line parallel to the longitudinal axes X1′-X5′ of the combing teeth T1′-T5′. The manner of gripping a parasite eradicating device having combing teeth 136 can best be understood as being similar to gripping a hair trimmer, such as those used for trimming hair close to the head. Thus, during combing, strands of hair pass in the combing paths H1′-H4′ formed by the spaces between the combing teeth T1′-T5′.

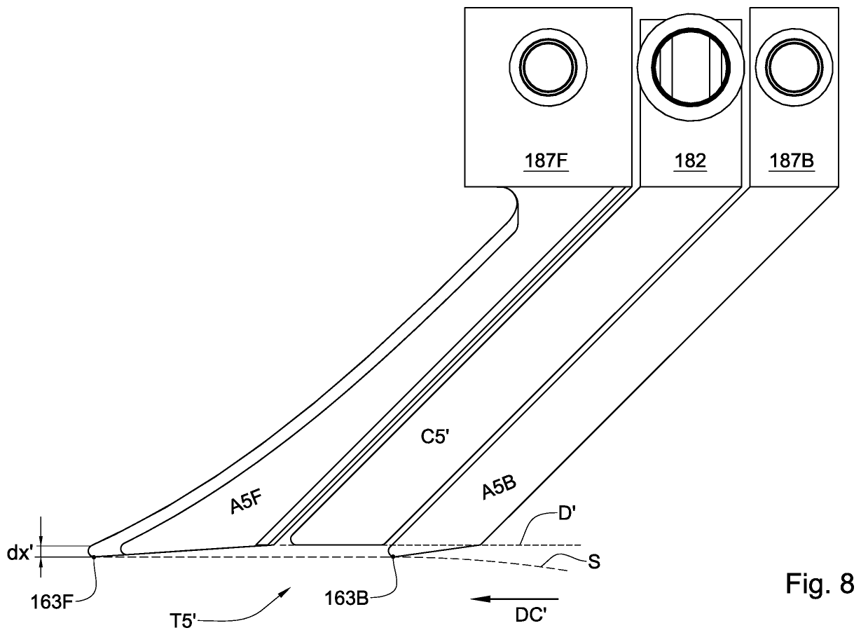

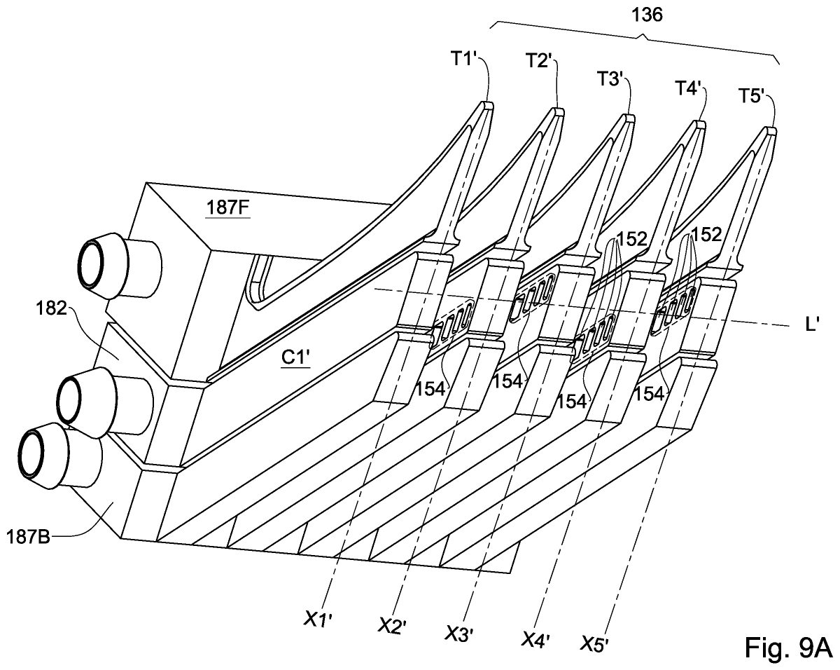

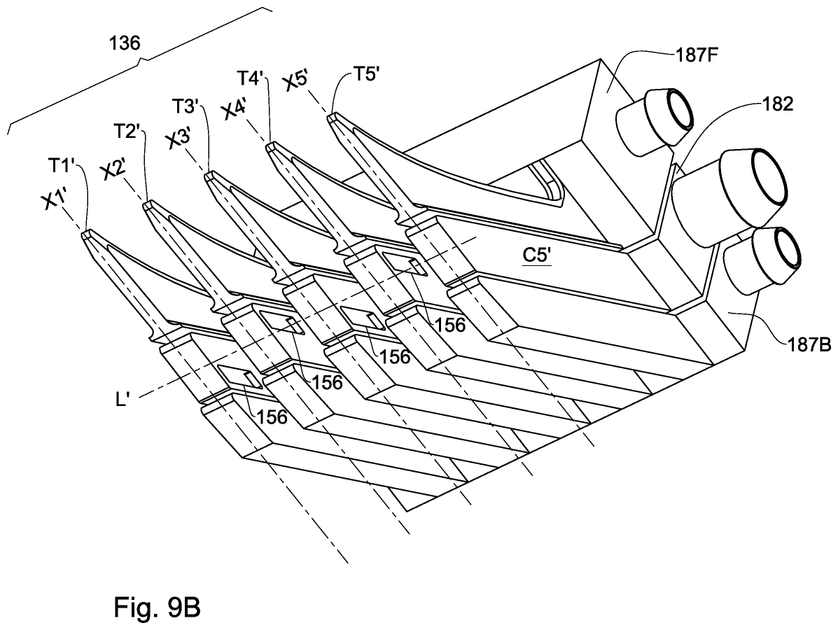

As in the example of the hand-held device 30 shown in FIGS. 1-6, each of the combing teeth T1′-T5′ is comprised of three components, which can be seen most clearly in FIG. 8 for the combing tooth T5′. The respective three components of each of the combing teeth T1′-T4′ can be seen in FIGS. 9A and 9B.

The central part of each combing tooth T1′-T5′ is a central operational combing tooth C1′-C5′, respectively, of which central operational combing tooth C5′ is visible in FIG. 8. Central operational combing teeth C1′-C5′ can be seen to be lined up in a row along their central axis L′, shown in FIGS. 9A and 9B.

As shown in FIGS. 9A and 9B, each of the high-temperature airflow outlet nozzles 152 and the inlet apertures 156 are disposed toward a distal end of the operational combing teeth C, such that, when the device head 138 (not shown) from which the set of combing teeth 136 extends, is brought as close as possible to the infestation area surface and the most distal contact points of exemplary combing teeth T′ make contact with the infestation area surface, the high-temperature airflow outlet nozzles 152 and the inlet apertures 156 will be located in the 0-6 mm zone from the infestation area surface.

Like the operational combing teeth C of the embodiment shown in FIGS. 1-6, operational combing teeth C′ of the embodiment shown in FIGS. 7-9B can be considered to be hot in temperature, as heated air flows through them and therefore, in order to protect the treatment recipient from discomfort due to heat, the operational combing teeth C′ are also prevented from coming into contact with the infestation area surface.

In the embodiment of hand-held device 10 shown in FIGS. 7-9B, as shown in FIG. 8, the distal end of operational combing tooth C5′ is prevented from coming into contact with the infestation surface S by the configuration of its adjacent auxiliary combing teeth, A5F and A5B, located to the front and back (with respect to combing direction R′) of the operational combing tooth C5′ respectively. It can be seen that when the set of combing teeth 136 is brought as close as possible to the infestation area surface S, contact is made with the infestation area surface S by the distal extremity points 163F and 163B of auxiliary combing teeth A5F and A5B respectively, while the distal end of operational combing tooth C5′ remains at the level indicated by the letter D′, at a distance dx′ from the infestation area surface S.

As can further be seen in FIGS. 7-9B, and most clearly in FIG. 8 which shows the shape of combing tooth T5′ in profile, combing teeth T1′-T5′, and the front row of auxiliary combing teeth A1F-A5F in particular, have an angled shape designed to effectively plow through, separate and lift the hairs in the infestation area S as they are combed, guiding the hairs into the combing paths H1′-H4′ and facilitating effective treatment of the 0-6 mm zone near the root of each hair as the penetration of the distal ends of the combing teeth T1′-T5′ into the 0-6 mm zone near the hair roots is optimized. The number of lice and nit targets intercepted and eradicated by the hot air stream emitted at the distal ends of the teeth, and therefore, the overall efficacy of the device, is thus maximized.

It can further be seen in FIGS. 7-9B, and most clearly in FIGS. 9A and 9B, that the high temperature airflow outlet nozzles 152 of combing teeth 136 are grouped in sets 154 of four nozzles 152 each. Four such sets 154 can be seen in FIG. 9A to be disposed on the left-facing sides of the operational combing teeth C2′, C3′, C4′, and C5′.

In FIG. 9B an inlet aperture 156 can be seen to be disposed on each of the right-facing sides of the operational combing teeth C1′, C2′, C3′ and C4′. It can consequently be understood that except for the operational combing teeth C1′ and C5′ disposed at the two ends of set of combing teeth 136′, each of the operational combing teeth C2′, C3′ and C4′ contain both a positive pressure hot air flow channel and a negative pressure (suction) hot air flow channel.

Positive air pressure for the flow of hot air is supplied to the positive pressure hot air flow channels in the operational combing teeth C1′-C5′, in a similar manner as that described above for the first embodiment, from a positive pressure manifold chamber 184 (not shown) inside of manifold 182.

Negative air pressure (suction) is supplied to the negative pressure (suction) hot air flow channels in the operational combing teeth C1′-C5′, in a similar manner as that described above for the first embodiment, from a negative pressure manifold chamber 186 (not shown) inside of manifold 182.

Manifold block 187F contains channels for the circulation of coolant fluid to the auxiliary combing teeth A1F-A5F, which are actively cooled. Manifold block 187B contains channels for the circulation of coolant fluid to the auxiliary combing teeth A1B-A5B, which are also actively cooled.

In an alternative embodiment of the set of combing teeth 136 illustrated in FIGS. 7-9B, central operational combing teeth C1′-C5′ could be actively cooled by actively cooled spacers such as actively cooled spacers 62 described above with respect to the embodiment of hand-held device 30 shown in FIGS. 1-6.

In yet another embodiment of the set of combing teeth 136 illustrated in FIGS. 7-9B, one of the combing teeth T1′-T5′ could be a motion sensing combing tooth, such as combing tooth T1, described above with respect to the embodiment of hand-held device 30 shown in FIGS. 1-6, and as illustrated in FIGS. 2, 3A and 3B.

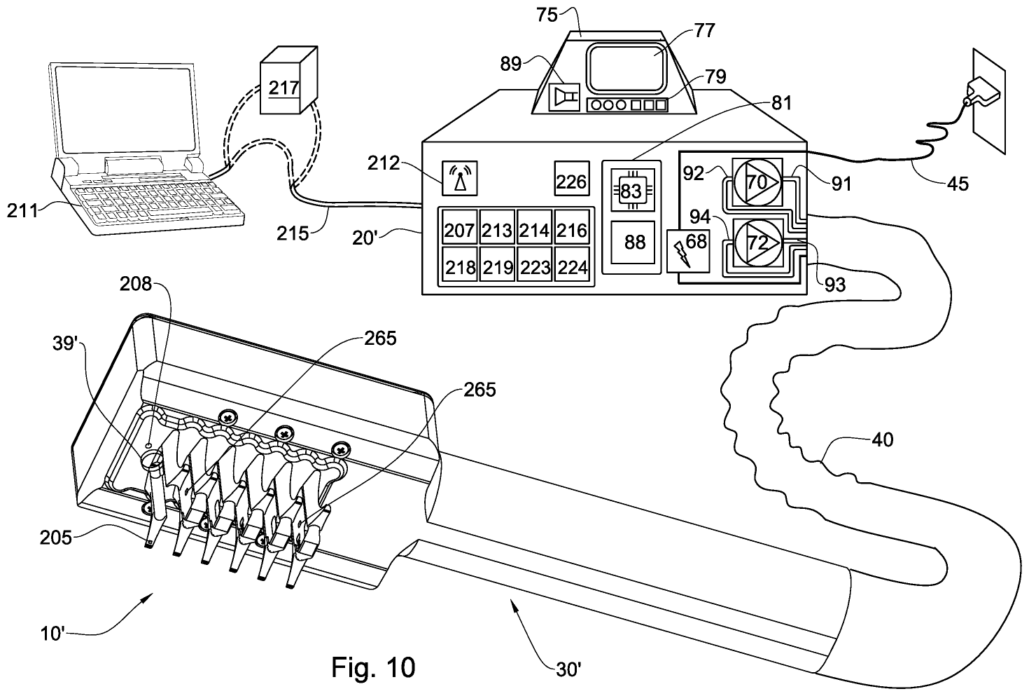

An alternative embodiment of the device 10 shown in FIGS. 1-6, device 10′, an example of which is illustrated in FIG. 10, comprises additional components which enable device 10′ to implement computerized vision and/or digital imaging technologies, including digital image analysis, utilizing imaging capabilities, data collection capabilities and processing capabilities. Device 10′ can then, with the addition of these capabilities, be used to detect the presence of parasites or nits in an area which is a potential infestation area. Device 10′ can further have additional functions facilitating the eradication of parasites and nits in an infestation area, owing to these additional capabilities, as will be described in further detail below.

Device 10′ can comprise an image recognition system such as computerized vision and/or digital imaging technologies. For example, as shown in FIG. 10, an exemplary device 10′ can comprise one or more image capturing device 205, an illumination unit 208, an optics assembly 39′ and a computing device 211 in order to provide imaging capabilities, data collection capabilities and processing capabilities needed to perform computerized vision and/or digital imaging technologies, including digital image analysis.

In accordance with the presently disclosed subject matter, the image capturing device 205 can be configured to capture images and/or video, the illumination unit 208 can be configured to illuminate a field-of-view of an area-of-interest for image acquisition or for video acquisition. The optics assembly 39′ can be used in conjunction with image capturing device 205 and/or illumination unit 208, to improve or to facilitate the illumination and/or the acquisition. An image capturing device can be configured at any one or more combing tooth, preferably near a distal end thereof.

Image data and/or video data that are acquired by image capturing device 205 can be temporarily stored in a local memory unit 207 within device 10′; and/or may be transferred or transmitted to a remote processing device, via a wired or wireless transmitter or transceiver 212. In FIG. 10, local memory unit 207 and transmitter or transceiver 212 are shown to be located in base unit 20′, however, they could alternatively be located in hand-held unit 30′.

Alternative optics assembly 39′ has additional capabilities beyond the motion sensing capabilities of optics assembly 39 of device 10 shown in FIGS. 1-6. For example, optics assembly 39′ can have a lens and/or other optics elements such as a mirror, a curved mirror, a concave mirror, a convex mirror, a focusing element, a defracting element, a prism, etc., which may enable efficient and/or maximum depth of field, and/or may otherwise facilitate high quality imaging by image capturing device 205.

Image capturing device 205 can be a camera or other imager, such as video camera, image-acquisition unit, CCD camera, CMOS camera, HD camera, camera-on-a-chip unit, or the like. Optionally, raw image data and/or raw video data, may be immediately transferred to an external memory unit and/or external storage unit and/or external processing unit, via a wireless link and/or wired link; optionally utilizing a wired transceiver and/or wireless transceiver (e.g., Wi-Fi transceiver, BlueTooth transceiver, ZigBee transceiver, or the like); optionally using one or more antennas or micro-antennas; optionally utilizing a short-term memory or buffer or cyclic buffer or accumulator unit to temporarily store image data and/or video data prior to its transfer to external unit(s); optionally utilizing a controller or processor or encoder or compression-unit to compress or encode the data (e.g., image data and/or video data) prior to such transmission. Image data and/or video data may be captured in a raw format and then may be locally compressed; or may be captured by the imager or by the camera in a manner that integrally outputs a compressed image type (e.g., JPG or PNG) and/or a compressed or encoded video format (e.g., MP4 or MP5 or MJPEG).

Illumination unit 208 can be any suitable illuminating element, such as a LED 3 or Organic LED (O-LED), other light-source or illumination component (e.g., a set of multiple LEDs), may be used to provide light and/or illumination; particularly, to generate light with automatic intensity adjustment to the cell interest, thereby enabling high-quality imaging and/or image acquisition and/or video acquisition by the image capturing device 205.

An optional data transfer cable 215 (e.g., a fiber or wired link) can be used to transfer the captured data, in raw format or in compressed or encoded format, to an external component or processor or processing unit 211 (e.g., laptop computer, tablet, smartphone, desktop computer). Optionally, instead of the data transfer cable or in addition thereto, a wireless communications transceiver may be included in device 10′, and may wirelessly transmit or upload such data (e.g., image data, video data) to external recipient devices. Optionally, data may be transferred from the device 10′, over wired links and/or wireless links, to a remote location or remote recipient, remote server, “cloud computing” server or repository, or other remote and/or local recipient devices.

Computing device 211 may receive and process the image data and/or video data, in real time and/or in retrospect; and may generate treatment feedback, treatment status updates, treatment options, treatment recommendations, proposed action items, progress indicators, and/or other indicators or feedback related to the extermination process and/or eradication process. Computing device 211 may be a local or a remote computing platform, and may be implemented by using, for example, a laptop computer, a desktop computer, a smartphone or tablet or “phablet” or smart-watch (e.g., optionally using a “mobile app” or mobile application), a remote server, a “cloud computing” server, or the like.

Optionally, an intermediary component 217 may facilitate the transfer of data from the device 10′ to the computing device 211; for example, by buffering data being transferred, or by providing short-term storage of data, or by providing other intermediate services (e.g., data compression, data encoding, data encryption) prior to or during data transfer.

As shown in FIG. 10, device 10′ can further comprise and image processor 213 which can locally process some or all of the image data and/or video data that is acquired by image capturing device 205. Image processor 213 may perform one or more image (or video) recognition algorithms, image (or video) analysis algorithms, image (or video) comparison algorithms, computer vision algorithms, and/or other processes which may be implemented via a computer vision module 218; and a lice treatment feedback/recommendations generator 219 may generate feedback and/or insights and/or treatment recommendations, which may then be conveyed to the user by one or more means; for example, by audible feedback (“beep” sounds, or pre-recorded speech message) which may be outputted via the audio speaker 89, by visual feedback (e.g., displayed on display unit 77, or via one or more LEDs or other visual indicator), and/or by transferring or transmitting such feedback or recommendations to an external device (e.g., transmitting the feedback or the recommendations via the transceiver 212, to be conveyed to the user via a smartphone or a tablet or a computer or other electronic device).

In a demonstrative example, image processor 213 may perform image analysis by comparing portions of an acquired image, to pre-defined images of a hair or a scalp or a louse or a nit, in order to determine or to estimate whether a region-of-interest comprises lice and/or nits, and in order to convey such feedback to the user.

Optionally, a lice gender detection module 214 may perform advanced image analysis, to determine whether a particular parasite that is imaged is a female parasite (which can hatch eggs or nits) or a male parasite (which cannot hatch eggs or nits); and to convey to the user suitable feedback, for example, indicating that a female louse (or several female lice) are detected in a particular region. This may be performed, for example, by image analysis or image comparison, between: (i) an image acquired by the image capturing device 205, and (ii) one or more reference images of a female louse, and (iii) one or more reference images of a male louse. Additionally or alternatively, this may be performed by a computer vision algorithm that searches for, and detects, particular visual features that characterize only a female louse and not a male louse. Some implementations may utilize, for example, one or more of the following differences between male lice and female lice: (I) in male lice, the front two legs are slightly larger than the other four; (II) male lice are generally smaller in size than female lice; (III) male lice are characterized by a pointed end of the abdomen and a well-developed genital apparatus visible inside the abdomen; (IV) female lice are characterized by two gonopods in the shape of a “W” at the end of their abdomen.

Optionally, a lice group detection module 223 may perform additional image analysis to determine that a particular region, which is imaged by a single image, or which is imaged across multiple images or within a video segment, contains therein multiple lice (or multiple nits, or multiple female lice); and to generate a suitable feedback to the user. For example, a lice counting module 216 may count the number of lice (and/or nits) that are detected within an image (or a set of images, or a video segment); and a lice infestation score generator 224 may generate a score (e.g., in a range of 0 to 10, or 0 to 100), indicating how severe the lice infestation is, for example, by comparing the counted number of lice and/or nits, per image (or per set of images, or per video segment, or for the area of a region-of-interest) to one or more pre-defined threshold values or ranges.

In some embodiments, the computer vision module 218 and/or the image processor 213 may perform other types of analysis, for example: automatic diagnosis of the existence and/or the severity of lice infestation; computer vision processes that utilize deep learning and/or machine learning, in order to improve the detection rate of lice and/or eggs as the device 10′ is utilized by the same user and/or by other users; detection of water, liquid, and/or other substances between or nearby the teeth or the outlets/inlets, which may interfere with the proper operation of device 10′; or the like.

In some embodiments, device 10 may utilize specific wavelength illumination and/or fluorescence and/or Ultra-Violet (UV) light and/or a combination of the above, in order to excite fluorescence characteristics of lice or/and nits to enhance or improve or highlight or emphasize the lice and nits with reference to its surrounding environment by improved illumination and/or to similarly improve the imaging and/or the image analysis and/or the computer vision processes; and/or may optionally utilize or create backlight shading effect(s) to achieve similar improvements.

In some embodiments, optionally, processor 83 may further track, log, or otherwise supervise the treatment along a time-line, such as, within single usage session and/or across multiple usage sessions (e.g., across multiple hours or days or weeks). Optionally, changes in lice infestation status may be automatically tracked, logged, stored, and reported to the user; and may also be reported, in some implementations, to a remote third-party recipient (e.g., a physician's computer; a hospital or clinic computer; a school nurse computer; a school district computer; a governmental authority computer; a health department computer; or the like).

In some embodiments, some or all of the components and/or modules that are shown in FIGS. 5 and 10 to be located in base unit 20 and 20′ respectively, can be located in hand held unit 30′, or alternatively, in an external unit wired to base unit 20′, or communicating wirelessly with hand held unit 30′ and/or base unit 20′. In other embodiments, some or all of the components and/or modules that are shown in in in FIGS. 5 and 10 to be located in base unit 20 and 20′ respectively, may be implemented externally to device 10 and 10′ respectively, as external components and modules that are external to device 10 and 10′, and/or as part of a computing device, tablet, smartphone, laptop computer, desktop computer, local server, remote server, “cloud computing” server or device, or the like. In some embodiments, some or all of the components and/or modules that are shown in FIGS. 5 and 10 to be located in base unit 20 and 20′ respectively, are implemented both locally within device 10 and 10′, either in the hand-held unit 30 or 30′ respectively, or in the base unit 20 or 20′ respectively as well as externally to (or remotely from) device 10 and 10′ or may be otherwise distributed among device 10 and 10′ and one or more other devices.

Optionally, a vibration module 226 may be included in device 10′, and may generate vibrations for one or more purposes and/or at pre-defined time intervals and/or triggered by one or more conditions. For example, vibrations may be generated at pre-defined intervals (e.g., every second, or every three seconds), to ensure that a hot-air treatment or other type of treatment is being employed towards additional vibrations due to the generated vibration.

Optionally, one or more of the combing teeth of the device, such as combing teeth T2-T6 of FIGS. 1-6, or combing teeth T1′-T5′ of FIGS. 7-9B, or another component of the device 10 or 10′, may comprise other modules or components for lice detection and/or lice eradication. For demonstrative purposes, there are shown two such treatment units 265 (FIG. 10), that may be part of teeth T2 and T6, respectively. For example, treatment units 265 may spray or eject a chemical agent that kills lice and/or nits, or may provide or generate an electric signal or electric voltage or electric current that can electrocute lice and/or nits, and/or may provide a mechanical force that kills (or breaks apart) lice and/or nits, and/or may perform a suction operation that sucks-away lice and/or nits, and/or may perform other suitable treatment operations, which may be performed in addition to or instead of the blowing of hot-air.

1-38. (canceled)

39. A parasite eradicating device configured for eradicating parasites by,
producing a high-temperature airflow;
directing the high-temperature airflow in a stream at an infestation area where parasites thrive; and
suctioning the high-temperature airflow away from the infestation area, such that the parasites are killed and eggs of the parasites are rendered nonviable by the heat of the high-temperature airflow.

40. The parasite eradicating device according to claim 39, further comprising:
a hand-held unit having a housing with at least two operational combing teeth extending from said housing, and configured such that a distal end of each of the at least two operational combing teeth comes into close proximity with a surface of parasite infestation area during a combing operation;
wherein at least one operational combing tooth of said at least two operational combing teeth has at least one high-temperature airflow outlet nozzle disposed on a first surface of said at least one operational combing tooth and is configured to emit a stream of hot air, and another at least one operational combing tooth of said at least two operational combing teeth has at least one airflow intake aperture disposed on a facing surface of said another at least one operational combing tooth, wherein said facing surface is at least partially facing said first surface, and said at least one airflow intake aperture is configured for suctioning said stream of hot air by a suction force generated in said device.

41. The parasite eradicating device according to claim 40, wherein said stream of hot air is organized into a concentrated stream of hot air by said suction force such that said concentrated stream of hot air flows forcefully across a separating space between said at least one high-temperature airflow outlet nozzle and said at least one airflow intake aperture.

42. The parasite eradicating device according to claim 40, wherein said stream of hot air has a temperature in a range between 80° C. and 120° C.

43. The parasite eradicating device according to claim 41, wherein said separating space is unobstructed.

44. The parasite eradicating device according to claim 41, wherein said separating space is the shortest possible distance between said first surface of said at least one operational combing tooth and said facing surface of said another at least one operational combing tooth.

45. The parasite eradicating device according to claim 40, wherein at least one of said at least two operational combing teeth comprises said at least one high-temperature airflow outlet nozzle and as said at least one airflow intake aperture.

46. The parasite eradicating device according to claim 40, wherein said at least one high-temperature airflow outlet nozzle is configured to emit said stream of hot air in a direction that is generally tangential to said surface of said infestation area.

47. The parasite eradicating device according to claim 40, wherein said distal end of at least one of said at least two operational combing teeth is configured with a spacer to maintain a space between said distal end and said surface of said infestation area when said hand-held unit is brought as close as possible to said surface of said infestation area.

48. The parasite eradicating device according to claim 40, wherein said at least one high-temperature airflow outlet nozzle is disposed near a distal end of said at least one operational combing tooth, and said at least one airflow intake aperture is disposed near a distal end of said another at least one operational combing tooth, such that when said distal end of said each operational combing tooth comes into said close proximity with said surface of said infestation area during said operation of said combing hair, said at least one high-temperature airflow outlet nozzle and said at least one airflow intake aperture are located within a range of 0-6 mm from said surface of said infestation area.

49. The parasite eradicating device according to claim 47, wherein said spacer is composed of a non-thermally conductive material.

50. The parasite eradicating device according to claim 47, wherein said spacer includes an auxiliary combing tooth, located adjacent to said operational combing tooth, so that when said hand-held unit is brought as close as possible to said surface of said infestation area, said auxiliary combing tooth comes into contact with said surface, and said distal end of said operational combing tooth is kept at a distance of said space between said distal end and said surface.

51. The parasite eradicating device according to claim 47, wherein at least a portion of said spacer that comes into contact with said infestation area surface is cooled by an active cooling system.

52. The parasite eradicating device according to claim 51, wherein said spacer includes a thermally conductive material.

53. The parasite eradicating device according to claim 51, wherein said spacer includes tubing having a thermally conductive material and forms part of a closed circuit through which coolant fluid flows and draws heat away from the tubing in order to reduce or eliminate hot contact with infestation area.

54. The parasite eradicating device according to claim 40, further comprising an air pump for generating an airflow for emitting the stream of hot air via the at least one high-temperature airflow outlet nozzle and for generating the suction force for suctioning said stream of hot air via the at least one airflow intake aperture.

55. The parasite eradicating device according to claim 54, further comprising a heating mechanism for heating the airflow.

56. The parasite eradicating device according to claim 54, further comprising:
at least one sensor;
a processor; and
a controller;
wherein said at least one sensor is configured to sense at least one parameter of operation of said device;
wherein said processor is configured to determine at least whether said at least one parameter of operation falls within a predetermined allowable range of values for said at least one parameter; and
Wherein said controller is configured to vary said operation of said parasite eradicating device in order to maintain said at least one parameter of operation within said predetermined allowable range.

57. The parasite eradicating device according to claim 56, wherein said at least one sensor includes at least one of a temperature sensor, a humidity sensor, a motion sensor, an accelerometer, a gyroscope, an orientation sensor, a positioning sensor, a compass, an illumination level sensor, or an acoustic sensor.

58. The parasite eradicating device according to claim 40, wherein at least one of the operational combing teeth is configured with a retraction mechanism disposed between said at least one of said operational combing teeth and said housing, wherein the retraction mechanism is normally biased such that a combing tooth thereof is in a normally extended position and such that the combing tooth retracts under pressure exerted upon the combing tooth by said surface of the infestation area at a start of said combing operation.
